(19) United States
(12) Patent Application Publication
(10) Pub. No.: US 2023/0404116 A1
(43) Pub. Date: Dec. 21, 2023

(54) SYSTEM AND METHOD FOR TREATING LIQUID BEVERAGE USING ELECTROMAGNETIC FIELD COMPRISING AC AND DC COMPONENTS

(71) Applicant: ECOSPEC NOVELTECH PTE LTD, Singapore (SG)

(72) Inventors: Hwee Hong CHEW, Singapore (SG); Marius Abella ASERON, Quezon City (PH); Xavier Alexen Roxas ASERON, Quezon City (PH); Jan Adrian Roxas ASERON, Quezon City (PH)

(21) Appl. No.: 18/038,133

(22) PCT Filed: Aug. 6, 2021
(86) PCT No.: PCT/IB2021/057255

§ 371 Date: May 22, 2023

Related U.S. Application Data
(63) Continuation of application No. 63/116,976, filed on Nov. 23, 2020.

(51) Int. Cl. A23L 5/30; A23L 2/38; A23F 5/24; C12H 1/16; A61K 41/00; A61P 3/04

(52) U.S. Cl. CPC A23L 5/30; A23L 2/38; A23F 5/24; C12H 1/165; A61K 41/0004; A61P 3/04; G01N 2021/3595

(57) ABSTRACT

The invention relates to a method and system for treating a liquid beverage using an electromagnetic field comprising AC and DC components to achieve antioxidizing, promoting fat/lipid burn metabolism, reducing alcohol toxic effect and improving drinking mouth feel and flavor. In particular, the invention relates to methods and systems for applying the DC biased time-varying frequency pulsating electromagnetic wave in a pulsating manner to the alcoholic and non-alcoholic beverages. The method and the system of the invention are able to result in various treatment effects for the beverages simultaneously.

### RELATED APPLICATION

This non-provisional application claims priority from provisional application No. 63/116,976 filed on Nov. 23, 2020, the disclosures of which are incorporated herein by reference in their entirety.

### FIELD OF THE INVENTION

The invention relates broadly a system and a method and for treating non-alcoholic and alcoholic beverages to simultaneously produce multiple beneficial treatment effects using an electromagnetic field comprising AC and DC components in pulsating manner. The beneficial treatment effects include antioxidizing, promoting fat/lipid burn metabolism, reducing alcohol toxic effect and improving drinking mouth feel and flavor etc.

### BACKGROUND OF THE INVENTION

With the increasing health consciousness and affluence of most population, beverages quality requirement is now not limiting only to taste. General public consumers want the beverages to be good for health or at least not harmful to their bodies, yet without compromising the tastes and quality of beverage enjoyment especially for alcoholic beverages.

On average, adult consumes more liquids than solid foods each day. Typically, each person consumes about 2 liters of liquid each day, far more than the solid foods consumed daily. The liquid mostly comes from beverages, which typically include water, tea, coffee, juices, wines, distillates and, etc.

For good health, nutritionists and medical practitioners always encourage consumers to consume more antioxidant-containing foods or beverages to neutralize the negative health impact of free radicals produced in the cell metabolism process and from external pollutant sources. The positive health effect of antioxidant is well documented and recognized in nutrition and medical practice. Many beverage makers therefore add in artificial vitamin C or other antioxidant plant contents into the beverages to claim the antioxidizing effect. However, despite the vitamin addition and the antioxidant contents in the beverages, many beverages we consume daily are still showing oxidizing when checked by an Oxidation Reduction Potential (ORP) meter with Ag/AgCl reference cell. It has been always a desire for a method or apparatus capable of converting a beverage from oxidizing to antioxidizing, so as to provide health benefits to the consumers without changing their beverage consumption habits.

Body fat reduction is another important parameter in keeping good health in check. Health-conscious consumers like to have foods or beverages that could help in reducing body fat without medication. Most working people nowadays are desk bound and sedentary. Their lifestyle hardly involves any physical work. Obesity is therefore an increasing problem for the modern society, and difficult to change. More precisely, the real health issue is about body fat/lipid reduction instead of mere weight gain. Unless people change their lifestyle and go for regular exercise to reduce their fat, this health issue due to excessive body fat will remain and continue to pose increasing health cost to the society. So far there are no unmedicated beverages available in the market that could help to burn body fat/lipid by consuming the beverages. It is desirable if treated beverages could promote more body fat metabolism at sedentary condition.

Beverages with the desirable health properties of antioxidizing and fat reduction effects as described above are advantageously palatable if not better than untreated beverages. If the taste and fragrance of the treated beverages are degraded or compromised, they will deter or cannot attract the consumers, hence the purpose of improving the wellbeing of consumers would fail.

It is also advantageous that the same treatment method and apparatus are applicable to both non-alcoholic beverages and alcoholic beverages, especially the high alcohol content distillates.

Alcoholic beverages in general are regarded as a kind of unhealthy beverage although some researches report that moderate wine drinking for certain people may have a positive effect on health. This is likely due to the positive health effect of antioxidant content in wine instead of alcohol. Nevertheless, while wines do contain antioxidants, the ORP for all wines is still showing oxidizing although they are less oxidizing than high alcoholic content distillates. It would be beneficial if wines and high alcohol content distillates can be treated from oxidizing to antioxidizing.

Alcohol is recognized physiologically to cause liver damage. When alcohol enters the blood stream through intestine wall, it will reach the first organ, liver. Liver enzymes break down alcohol into acetaldehyde and eventually to non-toxic acetic acid. In this process, although liver enzymes function to decrease the activation energy of alcohol to acid reaction, they still consume free energy in order to convert alcohol to non-toxic acetic acid. In the situation of insufficient body energy supply, instead of complete alcohol conversion to acetic acid, the conversion is incomplete and thus produces too much mildly toxic acetaldehydes. The accumulation of acetaldehydes may harm liver causing alcoholic fatty liver disease or other liver health problems. If additional energy, such as in the form of additional bond vibration energy, could be stored in the beverages after treatment, the treated beverages may promote and facilitate the alcohol to acid conversion and reduce the aldehyde accumulation, hence less harmful to liver.

Besides the demand for improved health effects for beverages, in order to attract alcohol beverages to consume healthier alcoholic beverages for minimizing negative health impact of alcohol, the treated alcoholic beverages preferably have good mouth feel and taste than untreated beverages, or at least no degradation in terms of mouth feel and taste takes place. This would attract and encourage people to take healthier beverages which yet have antioxidizing, fat metabolism promotion function and less harm to liver.

For alcohol beverages, better quality alcoholic beverages are generally referred to as reduced astringency for wines and reduced throat burning sensation for high alcohol content distillates. Presently, reduction of astringency for wines and reduced throat burning sensation for distillates are normally achieved by years or decades of aging in oak cask. Under the controlled oak cask aging storage environment, tannins monomer, wine protein, alcohol and polyphenol molecules undergo complex polymerization to reduce astringency and throat burning sensation. However, such polymerization naturally takes years of progression, and it will be revolutionary for the wine industry if an artificial treatment could reduce this process down to minutes even seconds.

Similarly, old distillates such as well-aged whisky in cask are much smoother or have less throat burning sensation compared with younger whisky. Such smoothness is very much appreciated by the consumers, and thus commands higher value. A 25-year-old whisky may be priced tens or hundred times higher than a 12-year or younger age whisky. But producing this smooth effect generally takes 10 years or more of good maintenance storage in oak cask. The same applies to other types of distillates. But once the distillates are removed from cask and bottled, the taste, throat burning sensation, or smoothness of the bottled high alcohol content distillates cannot be changed or improved further. In other words, high alcoholic beverages such as whisky cannot be aged further once taken out from oak cask. This is well acknowledged as an established fact in the distillates industry and trades. It is thus being contemplated that the bottled whisky or high alcoholic distillates allow for further reduced throat burning sensation to simulate the aging effect.

Throat burning sensation is usually not an issue for low alcohol content beverages such as wines or some Asian wines. But astringency of the young wines gives undesirable coarse rough mouth feel when consumed. Although some bottled young wines can be further aged in corked bottles, it takes years to reduce such astringency feel.

Astringency is also a typical issue for the beverages prepared from plant leave or fruits such as tea, coffee or herbal tea. To suppress such astringent feel, typically other tastes such as sweeteners or sour agents are added into non-alcoholic beverages to suppress the astringency feel but sugars or chemicals addition to the beverages is unhealthy. It is desirable for reduced astringency of the beverages without the need for unhealthy suppressing agents to maintain the same mouth feel and flavor.

Many efforts have been made to address the above-mentioned needs and desires for improved quality of alcoholic and non-alcoholic beverages. Nevertheless, they can achieve only one of the desirable properties instead of obtaining all in one go treatment. Moreover, all of them take a long time to produce just one of the effects.

Ultrasound treatment disclosed in U.S. Pat. No. 7,220,43962 utilizes ultrasound mechanical wave to accelerate breakdown or increase the cell wall permeability of oak, grapes, or other ingredients for faster binding of ingredients. But it does not have any function in antioxidizing the beverages or change the beverage ORP from oxidizing to antioxidizing. This treatment is primarily used for fast extraction of content from grapes, malt, oak or the ingredients in the beverages during wine or whisky manufacturing process but not after bottling. While the taste may be improved due to better extraction, burning sensation or astringency reduction is not the main treatment purpose.

Pulsed Electric Field (PEF) treatment disclosed in CN 12564210 applies a high electrical voltage in kV level to increase the cell wall permeability of oak, grain, grapes and other ingredients to faster extract the content in the beverage ingredients. The effect is similar to ultrasound treatment and sometimes these two methods are used in combination. The electric field so applied may be in a fixed frequency pulsating manner for improving the extraction efficacy. But it has no function or capability in changing the beverage ORP from oxidizing to antioxidizing, either.

Electromagnetic coil field treatment disclosed in CN 2305406 Y utilizes a high frequency electromagnetic signal to produce a magnetic field emitter through emitting post. To enable the emitter post to emit the signal, the frequency must be mega Hertz or in higher order. The circuit depicted in the patent does not generate a time varying frequency signal. In this prior art, the beverage is subjected to a weak external alternating AC fixed frequency field treatment. With this arrangement and according to the patent description, it can improve the taste of the beverage to some extent, but it takes hours for the effect to become noticeable. This method is also unable to convert the beverages from oxidizing to antioxidizing. The patent is also silent about the burning sensation and the astringency mouth feel reduction.

There are other technologies that teach to use time varying electromagnetic wave instead of fixed frequency wave as above, but these technologies still use inductor coils (such as US patent application no. US 2017/02552439 A1). They are also unable to change the antioxidizing property or to reduce the throat burn sensation, although it improves the astringency feel reduction to an extent.

Aeration treatment is another prior art method that utilizes air introduced by swirling, stirring or venturi eductor, the air is entrained into the beverages according to an inverse relationship of flow pressure and flow speed. With the air entrained, the aroma releasing rate is elevated to give better aroma nose senses compared with resting beverages. The released aroma affects and confuses the taste bud sensory system, thereby giving an illusion of taste improvement. But once the aroma molecules are fast released in the aeration process, the aroma will be significantly reduced shortly after aeration. The ORP of the beverages may change to more oxidizing in the process, and the burning sensation of high alcoholic beverages or the astringency of wines cannot be reduced.

Treatment of direct current (DC) electrolysis method is mainly and only used in producing alkaline water. The alkaline water is produced at the cathode side of separation membrane useful for DC electrolysis of water. A high pH will be generated due to the concentration of OH⁻ at the cathode area. This results in an increase in OH⁻ ions present in water and shifts the water ORP to antioxidizing, which however is accompanied by alkaline pH. The alkaline water is undesirable to the taste of beverages. Similarly, there are prior patent applications which teach the use of a very low DC voltage of less than 3 volt with milliampere current to generate oxidative free radicals at the anode side in order to produce taste change for wines only. The applications are silent about the effect on high alcohol content distillates, or about the ability to reduce the astringency, burning sensation or fat metabolism.

Treatment of direct addition of chemicals such as bicarbonates can produce a negative ORP shift effect, but it alters pH of the beverages and changes the taste as well. It has no effect on reducing the burning sensation of the high alcoholic beverages or reducing the astringency of wines. In general, the chemicals addition would change the chemical properties and tastes of the beverages, which is not desirable.

The invention provides a system and a method of beverage treatment that are capable of simultaneously producing desirable beneficial effects on the liquid beverages in one go irrespective of the types of beverages, including but not limited to antioxidizing, promoting fat burn metabolism, reducing alcohol toxic effect and improving drinking mouth feel and flavor.

### SUMMARY OF THE INVENTION

The present invention has been developed to fulfill the needs noted above and therefore has a principal object of the provision of a system and a method of beverage treatment for simultaneously providing multiple treatment effects for the beverages in a single treatment without compromising the tastes and quality of beverage enjoyment especially for alcoholic beverages.

Another object of the invention is to provide a system and a method for simultaneously providing multiple treatment effects for the beverages which is significantly more economical and convenient than the processes and systems in the prior art.

A yet further object of the invention is to provide a system and a method for simultaneously providing multiple treatment effects for the beverages which can be achieved instantly.

These and other objects and advantages of the invention are satisfied by providing a system for treating a liquid beverage using an electromagnetic field comprising alternating current (AC) and direct current (DC) components, comprising:
a device for generating a DC biased time-varying frequency pulsating electromagnetic wave comprising a biased DC component,
a power supply for supplying power to the device,
a treatment chamber containing a treatment liquid beverage,
one or more capacitive emitter pairs arranged in a predetermined pattern and placed to be in direct contact with the treatment liquid beverage, wherein each two adjacent capacitive emitters are spaced at a predefined distance and respectively electrically coupled with first and second output terminals of the device,
wherein the device is configured to apply the DC biased time-varying frequency pulsating electromagnetic wave to the treatment liquid beverage to generate a pulsating capacitive field having a field strength between the capacitive emitters, and
wherein a time-varying frequency and pulsating AC wave current comprising the biased DC component is provided in the treatment liquid beverage to produce a negative oxidation reduction potential (ORP) shift in the treatment liquid beverage whose pH substantially remains constant under a combined effect of the field strength of the pulsating capacitive field and the biased DC component.

According to the invention, the time-varying frequency and pulsating AC wave current and the DC component of the generated field may be provided to energize the treatment beverage such that one or more treatment effects are induced in the treatment liquid beverage. The treatment effects include but not limited to one or more of the following:
1) reducing throat burning sensation of the treatment liquid beverage,
2) reducing astringency of the treatment liquid beverage,
3) increasing body fat/lipid burn metabolism through consumption of the treatment liquid beverage;
4) reducing harmful toxin effect of the treatment liquid beverage if alcoholic component is present, and
5) facilitating ageing effect of the treatment liquid beverage if alcoholic component is present.

In some cases, a plurality of emitter pairs may be included and placed in the treatment chamber in a predetermined pattern and come in direct contact with the treatment liquid beverage, or the plurality of emitter pairs are mounted inline at a position where the emitters are in direct contact with the treatment liquid beverage. The emitters may be provided in the form of plates or rods arranged in parallel coupling electrically with the first and second output terminals of the device generator, respectively. Preferably, the emitters substantially extend over an entire length of the treatment chamber.

In some cases, the plurality of emitter pairs may be provided as intermediate discharge emitters which are arranged in rows in parallel to each other. Each of the emitters may have a part of emitter surface covered by a conductive coating, and the coated emitter surfaces being oriented in one direction and arranged between non-coated emitter surfaces. The benefit of the intermediate discharge emitters is to reduce power consumption and increase efficiency.

In one embodiment of the invention, the two adjacent capacitive emitters may be spaced at a short distance which allows for generation of the capacitance effect between the emitters. The distance may be less than 10 mm, preferably less than 6 mm.

In one specific embodiment of the invention, the device for generating a DC biased time-varying frequency pulsating electromagnetic wave may comprise an AC wave generator for generating an AC electromagnetic wave having a time-varying frequency at a desired sweeping time, and a DC biasing unit electrically coupled in series with the AC wave generator. The DC biasing unit may be configured to produce a DC output transmitted to the AC wave generator for generating the DC biased time-varying frequency pulsating electromagnetic wave comprising a biased DC component.

In another embodiment of the invention, the device for generating a DC biased time-varying frequency pulsating electromagnetic wave may comprise an AC wave generator for generating an AC electromagnetic wave having a time-varying frequency at a desired sweeping time, which is programmed to create imbalance time varying frequency wave form to generate the DC biased time-varying frequency pulsating electromagnetic wave comprising a net DC component.

Advantageously, the system of the invention may further comprise an ORP meter for real-time monitoring and measuring the ORP of the treatment liquid beverage, and a cooler used to cool down the treatment liquid beverage and/or a stirrer for homogenization of the treatment liquid beverage.

In order for determination of the degree of energization of the treatment liquid beverage, the system according to the invention may also comprise a FTIR spectrometer to scan multiple consecutive beverage samples taken from the treatment liquid beverage to obtain a plurality of FTIR spectra which are mapped and analyzed to acquire a maximum vertical variation of peak height of a same absorbance peaks so as to determine a degree of energization, preferably a degree of non-thermal energization of the treatment liquid beverage.

Preferably, the device for generating the DC biased time-varying frequency pulsating electromagnetic wave may be configured to generate the DC biased time-varying frequency pulsating electromagnetic wave comprising a biased DC component having one or more of:
a wave form selected from square, sine, rectangular or triangular wave form,
a frequency between about 100 Hz and 1,000,000 Hz preferably between 500 to 10,000 Hz,
a sweeping frequency between 1 Hz and 1000 Hz, preferably 10 Hz and 100 Hz, and
a maximum peak voltage of the biased DC component has less than half of max AC peak voltage.

Another aspect of the invention is to provide a method for treating a liquid beverage using an electromagnetic field comprising alternating current (AC) and direct current (DC) components, comprising the steps of:
generating a DC biased time-varying frequency pulsating electromagnetic wave comprising a biased DC component, and
applying the DC biased time-varying frequency pulsating electromagnetic wave to a treatment liquid beverage to provide a time-varying frequency and pulsating AC wave current comprising the biased DC component,
wherein the time-varying frequency and pulsating AC wave current comprising the biased DC component is transmitted to one or more capacitive emitter pairs arranged in a predetermined pattern and placed to be in direct contact with the liquid beverage so as to generate a pulsating capacitive field having a field strength between the capacitive emitters, thereby to produce a negative oxidation reduction potential (ORP) shift in the treatment liquid beverage whose pH substantially remain constant under a combined effect of the field strength of the pulsating capacitive field and the biased DC component.

According to the invention, the method may further comprise the step of measuring a conductivity of the liquid beverage for selecting the emitters adapted for the treatment of the liquid beverage, and/or the step of determining a degree of energization, preferably a degree of non-thermal energization, of the treatment liquid beverage, as a feedback control for the beverage treatment.

In one preferred embodiment of the invention, the determination step comprises:
taking a plurality of consecutive samples from the treatment liquid beverage,
obtaining a plurality of FTIR spectra which represent respectively the plurality of consecutive samples,
mapping and analysis of the plurality of FTIR spectra for a same absorbance peak, e.g. OH bond absorbance peak, to collect information on peak height of the same absorbance peak, and
determining a maximum vertical variation of the peak height based on the lowest and highest peak heights of the same absorbance peak in the plurality of FTIR spectra,
wherein the maximum vertical variation of the peak height reflects the degree of energization of the treatment liquid beverage.

In some cases, the method of the invention further comprises the step of real-time monitoring and measuring the ORP of the treatment liquid beverage as a feedback control for the beverage treatment.

In contrast to the processes and systems available in the prior art which are typically designed to solve a certain particular problem and are inadequate for solving multiple and interrelated problems, the system and the method of the invention are more flexible and able to solve multiple problems to obtain the desired beverage treatment effects simultaneously, which significantly reduces the size of systems and the treatment cost. According to the invention, the use of DC biased time varying wave to produce the pulsating capacitive field is the unique way to produce antioxidizing effect (i.e. the ORP shift in the negative direction) in the liquid beverages yet without producing any significant change in pH, because the beverages are simultaneously subject to the combined effect of the capacitive field and the pulsating AC and DC components. After the beverages are treated in this way, various treatment effects can be obtained effectively. Importantly, the beverages possess the REDOX (reduction/oxidation) energy which can be used for enzyme REDOX reaction, especially the REDOX energy can accelerate the conversion of alcohol to acetic acid in the alcoholic beverages.

The objects, characteristics, advantages and technical effects of the invention will be further elaborated in the following description of the concepts and structures of the invention with reference to the accompanying drawings. The drawings illustrate the invention by way of examples without limitation to the invention in any manner.

### BRIEF DESCRIPTION OF THE DRAWINGS

In the following detailed description, reference is made to the accompanying drawings. In the drawings, like reference numbers represent like parts throughout the various views.

FIG. 1 is a schematic view of an exemplary arrangement of a beverage treatment system which is constructed in accordance with a first embodiment of the invention, in which the emitters are in “small spacing emitter pair” configuration.

FIG. 2 is an alternative arrangement of the emitters constructed in accordance with a second embodiment of the invention, in which the emitters are in “intermediate discharge emitter array” configuration.

FIG. 3 is another alternative arrangement of the emitters constructed in accordance with a third embodiment of the invention, in which the emitters are in “large spacing emitter pair” configuration.

FIG. 4A is an exemplary arrangement of the device for generating a DC biased time-varying frequency pulsating electromagnetic wave comprising a biased DC component according to the invention.

FIG. 4B is the wave form generated by the arrangement shown in FIG. 4A.

FIG. 5A is another exemplary arrangement of the device for generating a DC biased time-varying frequency pulsating electromagnetic wave comprising a biased DC component according to the invention.

FIGS. 5B and 5C are respectively the pure wave form and the DC biased wave form generated by the arrangement shown in FIG. 5A.

FIGS. 6A and 6B depict a vertical variation in FTIR OH bond peak intensity of two different bulk water samples.

FIGS. 7A and 7B depict a vertical variation in FTIR OH bond peak intensity between treated and untreated whisky samples.

### DETAILED DESCRIPTION OF EMBODIMENTS

While this invention is illustrated and described in preferred embodiments, the system for treating a liquid beverage using an electromagnetic field comprising AC and DC components may be produced in many different configurations, sizes, forms and materials.

The term “liquid beverage” used herein may refer to non-alcoholic and alcoholic beverages in liquid state.

The term “capacitive emitter” used herein may refer to an element that is able to employ the superimposed time-varying frequency electromagnetic wave to energize the liquid beverage, with a pulsating capacitive field generated across a pair of capacitive emitters.

The terms “instant” or “instantly” used herein may refer to the time in producing significant treatment effects all in one go in typically between 0.5˜5 mins for one liter of beverage. The variation in time is largely due to the difference in beverage content including alcohol, tannins contents, and the ingredients etc.

The term “negative ORP shift” used herein may refer to the ORP of the liquid beverage that shifts to more negative ORP reading after subject to the treatment by the invention. For example, shifting progressively from +300mV to 0 mV to −200mV is a negative shift in ORP.

The present invention is capable of simultaneously and instantly producing various desirable treatment effects in one go irrespective of the types of beverages, which will be discussed herein below.

Even if the beverage is a very good quality aged alcoholic beverage without the burning and astringency sensation, certainly its ORP still requires to be oxidizing and it does not promote fat burning metabolism. The alcoholic beverage receiving the treatment of the invention will further make the good quality alcoholic beverage healthier.

The same and single one system of the invention may be suitable for treating different types of beverages (alcoholic and non-alcoholic beverages including water) with the various treatment effects achieved simultaneously and instantly. This will reduce the operation and manufacturing costs for beverage treatment.

It has been acknowledged for many years that the throat burning sensation of bottled distillates such as whisky cannot be changed further. The invention represents a breakthrough and can further improve the taste and reduce the burning sensation of the bottled distillates such as whisky, brandy, Chinese Mao Tai rice wines, etc.

One unique feature of the invention is to use the ORP as a feedback control parameter to produce the desirable beverage quality. With the use of ORP feedback control parameter for optimizing the beverage quality and the ability to treat bottled distillates and wine, it makes possible to create one's own distillates or wines or cocktails instantly at home or in food and beverage (F&B) outlet without waiting for years or decade of natural aging, or large oak casks with environment control storage. This becomes an innovative commercial solution to pubs, restaurants, or F&B outlets.

The invention is also able to maintain a very much longer shelf life of the beverages compared with the untreated beverages. This includes maintaining good antioxidizing property, burning sensation, astringency, and taste after treatment. Shelf life is an important consideration for home, commercial also for industrial applications.

For the wine receiving the treatment of the invention, if not fully consumed, the remaining wine, which is stored with a cork in the bottle without vacuum but kept in the fridge, still remains good taste for more than 2 months. This makes it very convenient for light wine beverages. If the same is applied to untreated wine, the untreated remaining wine can turn sour and become “spoiled” in just a few days.

The system of the invention is a small sized unit suitable for home use without occupying a large space or may be scaled up for commercial F&B outlets or for large scale industrial applications.

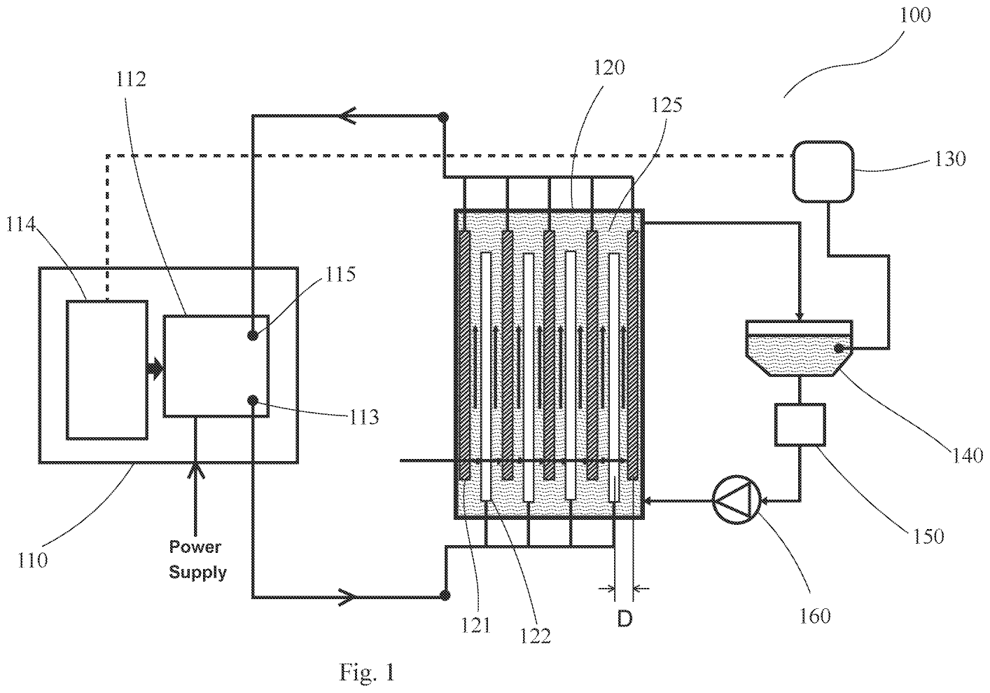

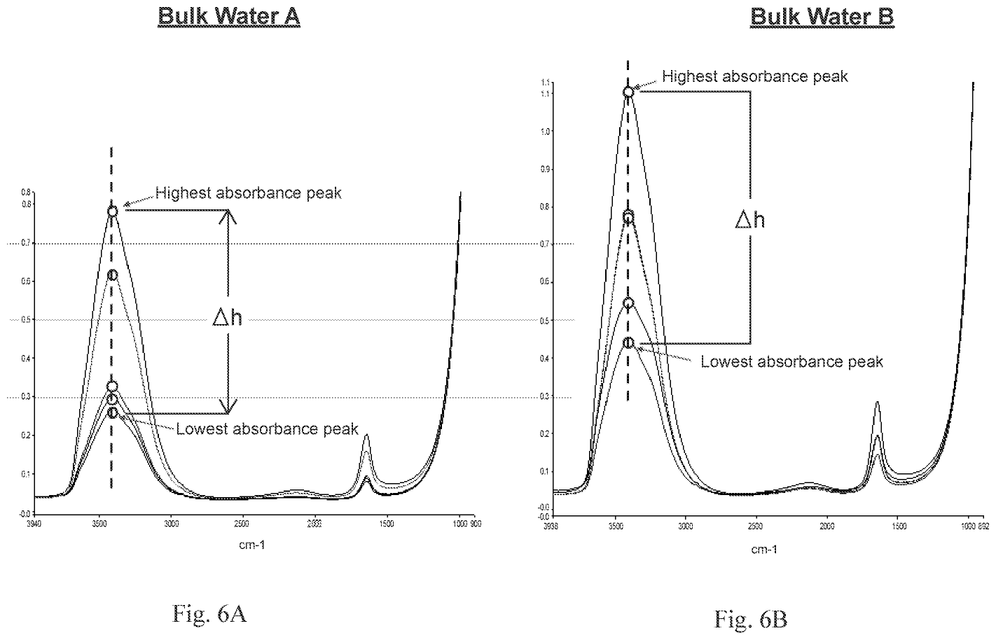

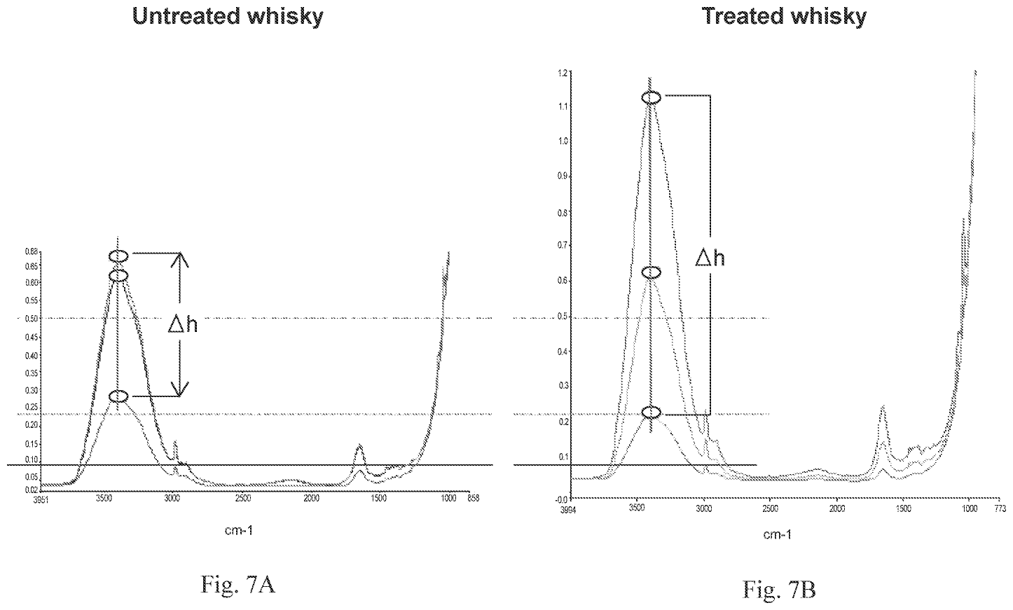

FIGS. 1 to 5C and the corresponding following description relate to methods and systems for treating liquid beverages to produce various treatment effects as mentioned above. FIGS. 6A to 7B and the corresponding following description relate to methods detecting and determining a degree of non-thermal energization of the treatment liquid beverage as a feedback control for the beverage treatment.

Referring now to the drawings, FIG. 1 illustrates a system 100 constructed consistent with a first embodiment of the present invention. In this embodiment, the system 100 is a closed loop circuit, and comprises a device 110 for generating a DC biased time-varying frequency pulsating electromagnetic wave. The device 110 comprises an alternating current (AC) wave generator 112 and a direct current (DC) biasing unit 114 which is a constant direct current power supply for supplying a DC component to the AC wave generator 112.

The system 100 further comprises a treatment chamber 120 for containing a liquid beverage 125 to be treated for the purposes of yielding various treatment effects as discussed above. The treatment chamber 120 may be open or closed. A plurality of capacitive emitter pairs are connected in parallel with the device 110 and immersed in the liquid beverage 125 contained in the treatment chamber 120. It is possible that the emitters may be mounted inline along a piping system circulating the liquid beverage 125 and are in direct contact with the liquid beverage 125. The capacitive emitter 122 of the emitter pair is electrically coupled with a first output terminal 113 of the device 110, and the capacitive emitter 121 of the emitter pair is electrical coupled with a second output terminal 115 of the device 110, as shown in FIG. 1. The emitters 121, 122 are spaced closely to bring a mutual effect therebetween such that the RLC (resistance, inductance and capacitance) components of the entire circuit are dominated by the capacitance effect to generate a capacitive field having a field strength across the emitters 121, 122 for treatment of the liquid beverage 125. The capacitance effect is essential for the negative ORP shift and/or other treatment effects.

The treatment chamber 120 may be made of a non-ferrous material, preferably a non-metallic material. If a metallic chamber is used, the internal surface of the chamber must be electrically isolated to prevent the absorption of electromagnetic field by the metallic materials.

The emitters 121, 122 and their arrangement in the liquid beverage 125 are important for obtaining the beneficial treatment effects. It is critical to generate a time varying electromagnetic field gradient which is very high, yet does not compromise the safe use by the operator or consumers. To resolve this issue, within the safe operating voltage of less than 60V, the emitters 121, 122 are preferably placed at a very small spacing of few millimeters (mm), which is called a “small spacing emitter pair” configuration. The smaller the spacing, the shorter the beverage treatment time. However, if a longer beverage treatment time is permitted, the spacing between the two adjacent emitters may be increased, but ensures the capacitance effect still exists. Further, the emitters 121, 122 shall be in direct contact with the treatment liquid beverage 125 and preferably extend over an entire length of the treatment chamber 120. The liquid beverage flows along the spacings of the emitter pairs so as to be treated by the pulsed capacitive field.

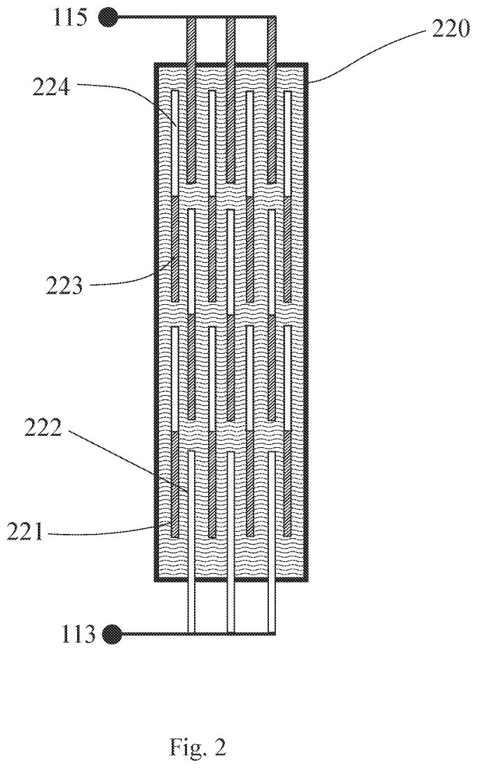

An alternative arrangement of the emitters is illustrated in FIG. 2, which is called “intermediate discharge emitter array” configuration. As illustrated, the plurality of emitter pairs are provided as intermediate discharge emitters 221, 222 arranged in rows in parallel to each other in a treatment chamber 220. Each of the intermediate discharge emitters 221, 222 has a part of emitter surface covered by a conductive coating, and the coated emitter surfaces 223 being oriented in one direction and arranged between non-coated emitter surfaces 224. The coated emitter surface 223 may only account for a half or any suitable length of the entire emitter surface, In the intermediate discharge emitter array configuration, the intermediate discharge emitters 221, 222 are not wire connected. Due to the lower energy required to discharge, the wave preferentially discharges only on the coated emitter surface 223. Such an intermediate discharge emitter array configuration allows to increase by many times the wave discharging and re-entering the emitters, unlike the small spacing emitter pair configuration where the wave current can only discharge once between the emitter pair. With the intermediate discharge emitters 221, 222, the same amount of wave current from the same emitter pair will enter and re-enter the emitter surface many more times according to the number of intermediate discharge emitters 221, 222. Therefore, to produce the same treatment effect, the amount of energy input is drastically reduced in this configuration.

The number of intermediate discharge emitters 221, 222 may be any number as long as the size of the treatment chamber 220 allows and the installation is not hindered.

Typically, the adjacent two emitters in the small spacing emitter pair configuration and the intermediate discharge emitter array configuration are spaced at a distance D of less than 10 mm, preferably less than 6 mm, making them generate a capacitive field suitable for treating the beverage having a conductivity of less than 500 μS/cm.

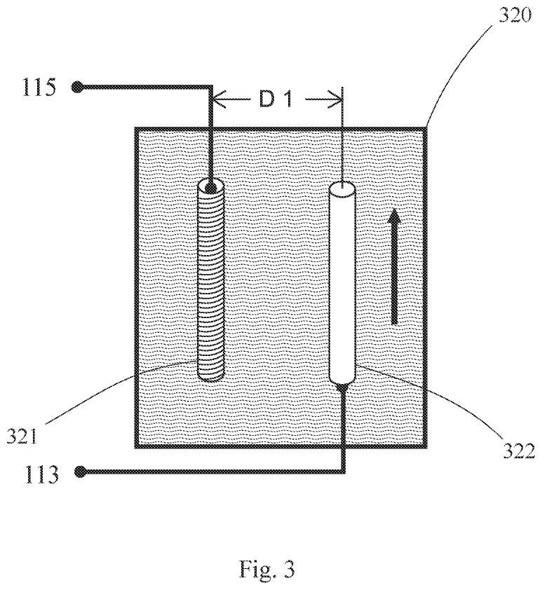

For beverages having a higher conductivity, a so called “large spacing emitter pair” configuration” is used, where the adjacent emitters are spaced at a relatively large distance, and the emitters are preferably designed to have reduced emitter surface area. An example of this configuration replacing the small spacing emitter pair configuration is shown in FIG. 3, where a pair of emitter probes 321, 322 spaced apart in a large distance D1 and contained in a treatment chamber 320 is illustrated. The distance D1 is more than 10 mm, preferably no less than 12 mm. With the reduced emitter surface area and the larger emitter spacing, the current flow between the emitters will be reduced and combined with the correctly sized constant current transformer to treat the beverages having a conductivity of up to 6000 us/cm.

According to the invention, the capacitive emitters is made of low consumption rate material such as gold, platinum coated titanium, niobium materials or any combination thereof. Other coating materials such as iridium oxides, ruthenium oxides, titanium oxides are suitable for use as a material of the emitters when they are placed in the treatment chamber. Boron doped diamond may be used for treating the beverages containing high chloride, including but not limited to coconut water, mineral water, salt added beverages etc. These kinds of beverages would produce hypochlorite or chlorine gas if using platinized or conductive metal oxide coated electrodes, due to DC ionic current in the beverages produced by the DC component of the time-varying frequency pulsating electromagnetic field. With the doped diamond electrode, the chlorine formation overvoltage is very high at the diamond electrode surface. This prevents the generation of chlorine or hypochlorite when treating the chloride containing beverages.

Semi-consumable materials such as graphite, graphene, silicon iron may also be used as a material of the emitters, as long as the slow dissolving materials is acceptable to the users and with no health issue.

Fast consumption materials such as magnesium, zinc or other metals may be used in the invention, if the dissolved magnesium or zinc ions are intended for specific consumption purpose.

The shape of the emitters 121, 122, 221, 222, 321, 322 may be in various forms of geometrical shapes in 2 or 3 dimensions to suit the space available in the device 110, including but not limited to mesh form, plates, bars, rods, concentric cylinders, coaxial tubes, single or multiple or repeating pairing layers.

The relative surface area between the emitters of emitter pair is adjustable to produce different effects for different types of beverages. For the beverages requiring more oxidation, a smaller surface area ratio of the two emitters is preferable, and vice versa.

The size of the emitters may be varied as a function of the load of the beverages and the targeted treatment requirements for the ORP, burning sensation and astringency reduction.

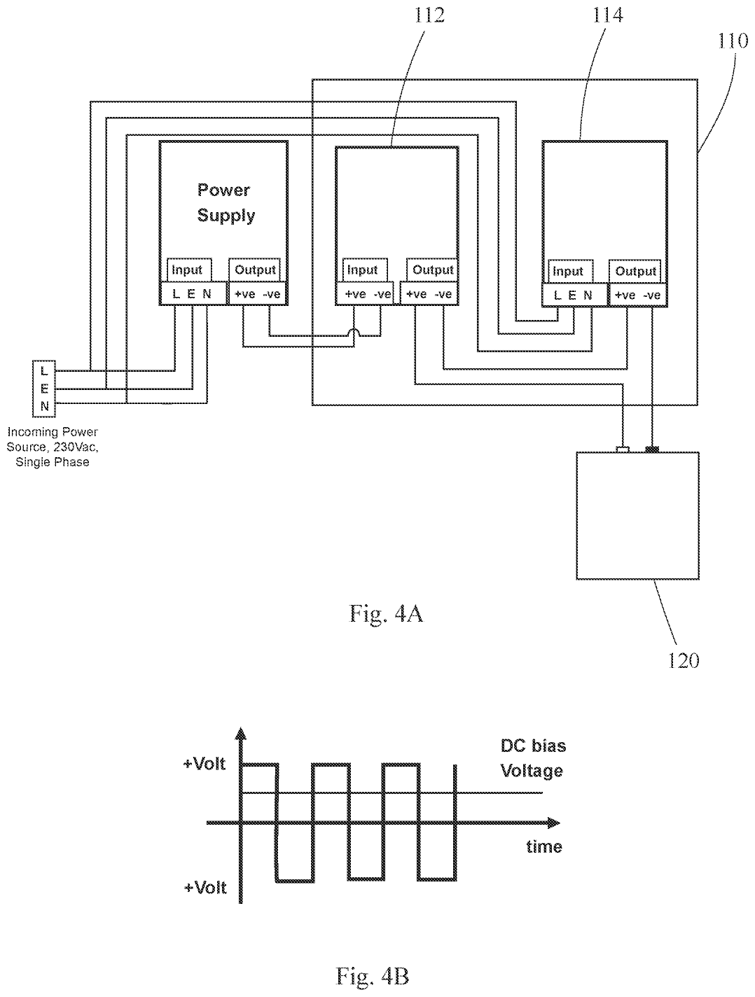

The AC wave generator 112 is electrically coupled with a power supply and configured for generating an AC electromagnetic wave having a time-varying frequency at a desired sweeping time. As illustrated in FIGS. 1 and 4A, the AC wave generator 112 is electrically coupled in series with the DC biasing unit 114. The DC biasing unit 114 is configured to produce a DC output with a predefined DC bias voltage. The DC output is transmitted to the AC wave generator 112 for generating the DC biased time-varying frequency pulsating electromagnetic wave comprising a biased DC component. In general the DC bias voltage is lower than the time-varying pulsating wave voltage and may be varied or fixed. The DC bias voltage is therefore adjustable to suit the different on-site treatment requirements.

Preferably, the frequency of the generated time-varying frequency electromagnetic wave used in the invention may be in the range of 100 Hz to 1,000,000 Hz, and preferably in the range of 500 Hz to 10,000 Hz; with the sweeping frequency being in the range of about 1 to 1000 Hz, and preferably in the range of 10 Hz and 100 Hz; and with a current r.m.s. output in the range of 0.5 amp to 300 amps for home to large scale industrial applications. The wave form generated by the device 110 of FIG. 4A is illustrated in FIG. 4B. It would be appreciated that the wave form can be square, triangular, rectangular, sinusoidal forms or even in random form, but square form is preferable.

The DC biasing unit 114 may be selected from a switch mode DC power supply, a rechargeable DC battery or AC-to-DC rectifier power supply. When the rechargeable DC battery is used as the DC biasing unit 114, an extremely pure DC output can be generated and is particularly suitable for some applications requiring an extremely pure DC source.

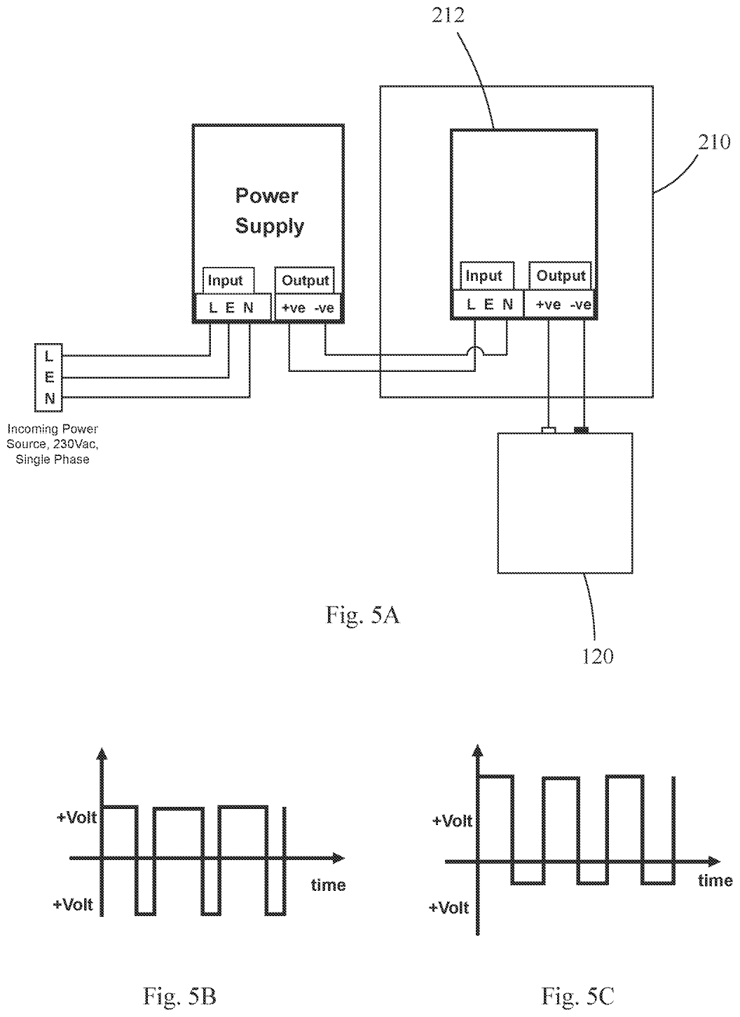

FIGS. 5A to 5C illustrate a possible alternative for the device 110. The illustrated device 210 comprises an AC wave generator 212 for generating an AC electromagnetic wave having a time-varying frequency at a desired sweeping time. In this embodiment, the AC wave generator 212 is programmed to create imbalance time varying frequency wave form to generate the DC biased time-varying frequency pulsating electromagnetic wave comprising a net DC component. FIG. 5B illustrates the pure AC electromagnetic wave form, and FIG. 5C illustrates the wave form comprising the net DC component generated by the device 210.

Another alternative for the device 110 may comprise an AC wave generator to drive one or more inductor coils for generating an AC electromagnetic wave having a time-varying frequency at a desired sweeping time, and a DC electrolysis means. In this embodiment, the inductor coils can be placed either inside or external to the treatment chamber, but the chamber must be non-metallic to prevent shielding of the electromagnetic field inside the treatment chamber.

Turning now to FIG. 1 again, the system 100 further comprises a beverage reservoir 140 for feeding the liquid beverage 125 to the treatment chamber 120, and an ORP meter 130 arranged for real-time monitoring and measuring the ORP of the liquid beverage 125 as an ORP feedback in order to obtain the best and most optimum treatment. The ORP meter is the type of Ag/AgCl reference cell. Once the negative ORP shift of the liquid beverage is slowing down and approaching the most negative ORP, it indicates that the optimum treatment is achieved and the treatment shall be stopped. Obviously, the exact optimum ORP reading will be different for different types of beverages and even between different batches of the same type of beverage. The optimum taste also differs from person to another. The users can therefore first establish the optimum ORP reading by tasting their preferred treatment results and then use the ORP readings for their subsequent treatment.

The ORP feedback can be manually controlled since the ORP reading of the ORP meter 130 is visible to the users. Alternatively, the users may set a desirable ORP end value and the system 100 may automatically stop the treatment when the predefined ORP value is reached. In addition to the ORP feedback, the treatment can also be timer-controlled for easier use when treating a large quantity of the same beverage. In order for better effecting the ORP feedback or timer-control of the treatment, a circulation pump 160 is provided for circulating the liquid beverage 125 between the beverage reservoir 140 and the treatment chamber 120 in the system 100 for better mixing and circulating the beverage in the treatment chamber 120.

A stirrer (e.g. magnetic stirrer, not shown) may be provided in the treatment chamber 120 and/or in the beverage reservoir 140 for facilitating homogenization of the treatment beverage 125. The magnetic stirrer works by rotating a small magnetic bar in the beverage when it is placed inside and subject to the rotating magnetic field produced by an external rotating polarities induction coil arrangement. Other methods known in the art may be adopted to produce the rotational magnetic field by alternating firing of multiple inductor coils. For example, it can be a fixed DC or AC current firing. The effect of using the rotating magnetic field can help to better mix the beverage, and the rotating magnetic field can also help in producing the water clusters. This can produce a weak negative antioxidizing shift in the ORP and the stirring effect also improves the aroma release from the beverage. The magnetic stirrer is therefore useful if incorporated in the treatment system of the invention.

One feature of the invention is the determination of a degree of energization of the treatment liquid beverage. The energization degree of the treatment liquid beverage represents an indication whether the beverage is sufficiently treated to the desirable energy level, and therefore may serve as the feedback control for the beverage treatment. FIGS. 6A and 6B illustrate schematically the basic principle for determination of the energization degree (preferably non-thermal energization degree) of water using samples of bulk water A and bulk water B.

The determination of the non-thermal energization degree of bulk water A comprises taking 5 consecutive samples from bulk water A, followed by consecutive scanning of these 5 samples to obtain their respective FTIR spectra. All these FTIR spectra are then mapped and analyzed for their OH bond absorbance peak to collect information on OH bond absorbance peak height. The vertical variation Δh in the lowest and highest peak heights of the OH bond absorbance peak of the 5 FTIR spectra is determined, and this parameter Δh represents the OH bond vibration energy active/dynamic state of the water. The same procedures are applied to bulk water B, and the vertical variation Δh in the lowest and highest peak heights of the OH bond absorbance peak is depicted in FIG. 6B.

It is clear that bulk water B has a greater vertical variation Δh than bulk water A, suggesting that the OH bond vibration energy of bulk water B is higher than that of bulk water A. The larger fluctuation between the lowest and highest peak heights, the more active dynamic change in OH bond attraction forces and interaction between the water molecular bonds instantaneously at different parts of the water.

FIGS. 7A and 7B depict the FTIR spectra of whisky before and after the treatment by the system 100 of the invention, which are obtained by following the above determination procedures for bulk water.

Specifically, three samples of 1 ml pipette drop taken from the untreated whisky without any treatment or changes in external factors such as temperature are collected and scanned by a FTIR spectrometer. Each of the three samples of the untreated whisky exhibits a different OH bond absorbance peak which is depicted in the left graph of FIG. 7A. After the whisky is treated by the pulsating field and the DC component of the invention system 100, three samples of 1 ml pipette drop taken from the treated whisky are collected and scanned by the FTIR spectrometer. Each of the three samples of the treated whisky also exhibits a different OH bond absorbance peak which is depicted in the left graph of FIG. 7B. Again, the vertical variation Δh in FIGS. 7A and 7B is determined respectively. It is obvious that the vertical variation Δh in FIG. 7B is much greater than the vertical variation Δh in FIG. 7A, suggesting that the whisky is indeed energized significantly by the system 100 of the invention.

In addition to OH bond, there are other bond peaks behaving in a similar manner but less obvious than OH bond peak because their bond vibration energy states may not be as dynamics as the OH bond vibration. Nevertheless, the same mapping and analysis principle and method can still be applied to determine the energization of other types of bond such as C═O, C—H, C═H etc.

The determination method for the energization degree of the beverage by mapping and analysis of OH bond excitation with FTIR spectroscopy may be used externally as one standalone unit or integrated into the whole treatment system 100 of the invention for the feedback control measurement of non-thermal molecular bond energy excitation. Alternatively, multiple FTIR spectrometers may also be used to scan multiple samples simultaneously, and they may also be modified so as to suit in-line analysis measurement with auto samplings.

With reference to FIG. 1 again, a cooler 150 is mounted in the system 100 in order to resolve the issue of heat generation during the treatment process especially in high conductivity situation, where the temperature of the liquid beverage 125 may rise, and overheating may take place if the treatment is unattended. The rise in the beverage temperature especially for wines is undesirable, and the cooler 150 is provided to cool down the liquid beverage 125 after treatment. The cooler 150 may be mounted such that it cools down the beverage stored in the reservoir 140 or the beverage flowing in the piping system. With the limited space available in the home use device, a compact thermoelectric cooler may be chosen to cool down the beverage after treatment.

FIG. 1 illustrates the system 100 where the liquid beverage 125 is treated in a flowing manner. Particularly, the pump 160 and associated piping are mounted to circulate the liquid beverage 125 contained in the treatment chamber 120. It is also possible to treat the liquid beverage 125 under static condition by use of the system of the invention. In the static case, no pump or associated piping system are required, and the static liquid beverage 125 is contained in the treatment chamber 120 to receive the treatment. The ORP meter 130 is mounted to the treatment chamber 120 onto which a cooler jacket may be sleeved, or other cooling system suitable for cooling the treatment cooler may be adopted, when necessary.

### Treatment Parameters Related to Conductivity of Beverages

This invention has the ability to treat a vast variety of beverages in the range from very low conductivity vodka with a conductivity of 10 μS/cm, to whisky and brandy with a conductivity of 30 to 60 μS/cm, water/tea/coffee with a conductivity in hundred range, to wine with a conductivity of 2000 to 4000 μS/cm and to coconut water with a very high electrical conductivity of 6000 μS/cm. With such high variation in conductivity range and due to the close proximity between emitter surfaces and the DC components in the time varying capacitive field, the current flow variation between the emitters can be different by up to 600 times if using the same treatment chamber and emitter configuration. According to the invention, the conductivity of the beverages can be either manually classified according to the types of beverages, which is particularly suitable for domestic use, or by manually measuring the conductivity readings. It can also be automatically classified by using an electrical conductivity sensor.

Typically, the beverages with the conductivity of less than 500 μS/cm can be grouped as one group using the treatment chamber in the small spacing emitter pair configuration as shown in FIGS. 1 and 2 designed for low conductivity beverages. For the beverages with the conductivity higher than 500 μS/cm, they may be treated in the treatment chamber in the large spacing emitter pair configuration according to FIG. 3.

However, even for the conductivity in the range of 5 to 500 μS/cm, the variation in the current for different types of beverages is still very large. The invention resolves this issue by using a constant current power supply which automatically regulates the output voltage to maintain a constant current supplied to the AC wave generator. With this constant current supply, it will not fuse the wave generator and ensures the constant treatment even when the beverage conductivity is varying. Moreover, using a larger size constant voltage power supply is possible as long as it can withstand the maximum current produced in the highest conductivity treatment.

A suitable power supply useful in the invention is preferably in the following ranges for effective coverage for the treatment functions.
Constant voltage supply of less than 60V may be used if the electrical conductivity of the varieties of beverages are restricted to only a fixed narrow range and the fixed power supply voltage and the current load so produced do not overload the power supply and over treat the beverages.
Preferably the constant current power supply is used for controlling the current output not exceeding the preset limit due to changes in electrical conductivity of the beverages.
The power supply can be pulsed switching rectifier type or inductor coil winding transformer rectifiers using silicon full wave or half wave bridge rectifier, or simple DC power supply from dry or wet battery.
The power supply can be air cooled or oil cooled.

It is possible for the invention to configure multiple treatment chambers in combined use with multiple emitters configurations discussed above, and this combination can be provided in one system of the invention to share the same and single one device 110 for generating the DC biased pulsating wave. This is particularly advantageous for the scenario where many different types of beverages with large variation in electrical conductivity require to be treated.

### ORP Feedback Control

As discussed above, the ORP readings are used to confirm the optimum treatment results for the beverages, e.g. wine and high alcoholic content distillates according to the invention. The optimum treatment results mean the following:
The ORP value of the beverage is close to the most antioxidizing.
The burning sensation is reduced to almost the best level.
The taste and aroma of the beverage are well opened providing a fuller spectrum of taste and fragrance.

The beverage receive the treatment of the invention to cause more polymerization of water, alcohol, aldehyde, acetic acid, ester molecules, more electron donating polymerized and clustered molecules are formed. The polymerized and clustered molecules with reduced unpolymerized alcohol molecules are the main contributors to the reduction of throat burning sensation and the reduction in astringency. These polymerization processes especially the water clusters rearrangement produce more electron donating clustering polymers hence the ORP reading of the beverages is showing more negative or antioxidizing.

Typically, in the initial stage of treating the alcoholic beverages, the ORP of the beverage may shift very quickly to the antioxidizing negative ORP reading. However, if the treatment is continued, the continuing OH⁻ and H⁺ ions production by the DC component may also produce oxidation by products including acetic acid, esters etc. At this stage, provided there is sufficiently active high bond vibrational energy polyphenols, tannins, alcohol etc., to polymerize with the acid, otherwise the acid will become too excessive. Excessive acid can affect the negative ORP shift rate and slow down the negative shift. After reaching the most negative ORP potential, the ORP will start reversing and moving into the positive oxidizing ORP direction. At this stage, the excessive acid production can change the taste of the alcoholic beverages to either become too flat or too sour showing typical characteristics and taste of an overaged wine or an overexposed-to-oxygen beverage.

In consideration of the above treatment behavior, the ORP meter 130 is arranged for real-time monitoring the ORP change. Once the negative ORP shift of the beverage is slowing down and approaching the most negative ORP potential, it indicates that the optimum treatment by the invention is achieved and the treatment process shall be stopped. Obviously, the exact optimum ORP readings vary for different types of beverages and even between different batches of the same type of beverage. The optimum taste also differs from person to another. Users can therefore first establish the optimum ORP readings by tasting their preferred treatment result and then use those ORP readings for their subsequent treatment of the beverages without the need to taste the beverage again.

As mentioned above, the ORP feedback can be manually controlled since the ORP readings are visible to the users. Alternatively, the users may set a desirable ORP end value and the system 100 of the invention may automatically stop the treatment process when the optimum ORP value is reached. In addition to the ORP feedback, the treatment can also be timer-controlled for easier use when treating a large quantity of the same beverage. Therefore, the ORP feedback allows to obtain the best and most optimum treatment of the beverages.

Determination of Non-Thermal Excitation of Molecular Vibration Energy of Beverages The energy is known to exist in many forms, but all forms are under the framework of Gibbs Free Energy. Enthalpy (thermal) and Entropy (non-thermal) energies are the two components in the concept of Gibbs free energy. The thermal energy is the most common form, which can be detected and measured by thermometers, but this is not the case for the non-thermal entropy energy. Entropy is a non-thermal energy characterized by the excitation of the molecular bond energy. Entropic energy involves bond vibrational and rotational energy including chemicals/concentration gradient energy, etc. In the cases of water or water containing beverages, OH bond vibration energy is the critical excitation, yet such excitation cannot be detected by thermal or temperature measurement. The existence of entropy affects many aspects of chemical reactions, especially in many bio-chemical processes/reactions.

Many research and technical work uses Fourier Transformed InfraRed (FTIR) spectroscopy to detect the bond vibration energy state of water and other substances, but they are silent about the methodology of how to apply it correctly in determining the dynamic bond vibration state of static bulk liquid, or when the bulk liquid is subject to an external excitation such as the entropy treatment of the present invention.

Although a conventional FTIR spectrometer can detect the OH bond absorbance peak, but the conventional teaching and method of using one single scanning of FTIR spectrum is unable to determine whether the OH bond vibration energy of the liquid sample is already energized or has already attained its maximum energy level.

In some applications especially for large scale industrial applications of beverage treatment, it is necessary and advantageous to detect whether the beverages are sufficiently treated to the desirable energy level serving from the perspective of entropy energy.

The system and method of the invention advantageously detect and determine the degree of energization of water or beverages, which can be used either as the treatment feedback control or for determining whether the treatment has achieved its desirable treatment level. Particularly, the invention teaches to map and analyze a plurality of FTIR spectra of samples obtained from the same beverage for the same absorbance peak to detect the excitation state of the bond vibration energy, and more critically, to detect and measure the level of the non-thermal excitation, as discussed above.

In the prior art technical research papers and publications, one sample is usually used and depicted in the FTIR absorbance or transmittance graph with only one single OH peak at the +/−3300˜3600 wavenumber range for this determination purpose. The conventional teaching of single line/single peak representation of FTIR spectrum typically shows only one spectrum line for each temperature as a theoretical or conceptual representation, and it cannot represent the actual OH bond vibration energy state, nor reflect the activeness or the dynamics of the sample. No teaching of whatsoever is used in the beverage industry or any prior art regarding the mapping method of using the vertical peak fluctuation in FTIR curve spectra. The prior art is also silent about using consecutive scanning of multiple samples taken from the same bulk liquid for detection of non-thermal energization of water or beverages. Rather, the conventional technologies teach that the OH peak of water can be broadened due to the changes in D₂O/H₂O concentration, or the OH peak can produce only one single response if being subject to one external input. But there is no such concept or teaching prior to the invention that even without the external state/energy changes, the water OH bond peak itself is so unstable, and therefore cannot be represented by one single absorbance peak value or single FTIR scan.

The invention proposes to map out the OH bond vibration energy state of water or beverages in the plurality of FTIR spectra either in static conditions or when being subject to external energy excitation. The OH bond peak absorbances of the water or beverage samples even when remaining at static condition can vary when multiple consecutive samples are taken from the same bulk water for the FTIR spectra scan, indicating the unstable nature of entropy energy since the water OH bond peak absorbance value is unstable and fluctuating. This method plots the trails of the OH peak intensities. When more consecutive samples are scanned by FTIR, more peaks will be detected and mapped. By plotting the trails of the peak intensities, usually a linear vertical line of the peaks can be observed. Or in some different excitation methods, a contour/boundary of the peak points can be mapped out if the peak trails are in more random order.

The vertical variation in absorbance peak height/intensity can reflect the change of the vibration states of the bond vibration energy. To establish the vertical variation in absorbance peak height, at least two samples are needed to locate the upper and lower absorbance peak height limits. More than two spectra would be preferred and more than five spectra peaks are more preferable to be taken to have a more accurate variation determination. After the first few peaks are obtained, if the subsequent FTIR scan spectra peaks all fall within the highest and lowest limits established by the first few samples, it indicates that the OH bond energy states of the water or beverages for determination is fluctuating within the range defined by the highest and lowest limits. More precisely, the water bond energy dynamics states shall be represented by a range of absorbance peak intensity value instead of a single peak intensity value. The greater the vertical variation in absorbance peak, the larger the fluctuation of the water and beverages and the higher active/excitation state the water and beverages have. In general, more than five spectra scan are adequate to determine the highest and lowest absorbance limits. But more samples always provide more accurate results.

Now turning to FIG. 6, if only one spectrum from each of bulk water samples A and B is taken, an incorrect interpretation may be acquired that the entropy energy state of bulk water sample A is higher than bulk water B. But in accordance with the determination method of the invention, it becomes clear that bulk water B has a higher OH bond vibration energy and in more active/dynamic states than bulk water A, due to the larger peak variation of bulk water B in vertical direction.

It should also be appreciated that the absorbance peak value of bulk water B is higher than bulk water A, but this only represents the higher absorbance of IR wave, and does not necessarily mean bulk water B is in more active/random vibration state than bulk water A. Only a larger fluctuation defined by the highest and lowest absorbance peak heights can reflect the more active dynamic change in OH bond attraction forces and interaction between the water molecular bonds instantaneously at different parts of the bulk water.

By measuring the amplitude change in OH bond absorbance peak, it is possible to establish a baseline for the untreated water. The absorbance peak variation of the untreated water signifies that the OH bond absorbance peak variation exists even without any energy input or without the change of surrounding state. This is different from the conventional teaching that only one OH bond absorbance peak value represents the whole bulk water. Obviously, more samples such as pipette drops are taken and tested, the better will be the establishment of the OH bond absorbance peak fluctuation amplitude range.

The OH bond peak mapping and analysis method discussed above to determine the level of excitation of bond vibration energy can be applied to different types of liquid beverages including ultrapure water, distilled water, tap water, ground water, seawater, alcohol drinks and many other types of solutions and liquids. This method for determining the energy degree of the liquids is particularly useful for the invention to effectively treat the beverages.

It has been surprisingly found that the system and method of the invention are capable of simultaneously and instantly producing various desirable beneficial treatment effects on beverages in one go irrespective of the types of beverages. The treatment effects include but not limited to the following:
1) producing a negative ORP in the treatment liquid beverage whose pH substantially remains constant,
2) reducing throat burning sensation of the treatment liquid beverage,
3) reducing astringency of the treatment liquid beverage,
4) increasing body fat burn metabolism through consumption of the treatment liquid beverage;
5) reducing harmful toxin effect of the treatment liquid beverage if alcoholic component is present, and
6) facilitating ageing effect of the treatment liquid beverage if alcoholic component is present.

### Producing the Negative ORP Shift

Oxidation Reduction Potential (ORP) is known as a parameter demonstrating the ability of chemical/biochemical systems to oxidize (lose electrons) or reduce (gain electrons). A positive value indicates an oxidized state, whereas a negative value indicates a reduced state.

To create the negative ORP shift in even low conductivity alcoholic beverages, the beverages are subject to the combined effect of the pulsating capacitive field and the biased DC component applied by the system of the invention.

Firstly, the treatment liquid beverage is exposed to the pulsating alternating AC time varying electromagnetic field to vibrate the O—H bond of water, alcohol, polyphenols, and/or other molecules containing O—H bond, hydrogen bonds or polar bonds. With the correct time varying frequency range, duty cycles, applied voltage and current in combination, such bond vibration becomes activated and is detectable by the larger amplitude change in the O—H bond vibration absorbance peak fluctuation in FTIR (Fourier Transform InfraRed) spectroscopy. With the high bond vibration, water molecules, poly phenols, alcohol molecules clustering arrangement are re-arranged, so as to accelerate polymerization of such hydrogen bonded and polar bonded compounds. In other aspects, the free energy of the beverage is elevated. Correspondingly the hydration encapsulation behavior of H⁺ and OH⁻ ions by water clusters are modified. For water and alcohol, this can be written as (H₂O)ₙ⁻, or (C₂H₅OH)ₙ⁻ denoting the stronger electron donating tendency of water clusters (H₂O)ₙ or alcohol clusters (C₂H₅OH)ₙ, or the mixture of ((H₂O)_{n′}+(C₂H₅OH)ₙ_{^{−}})ₙ clusters and negatively charged ((H₂O)_{n′}+(C₂H₅OH)ₙ_{^{−}})ₙ clusters, in which n is any numeric integer from 1 to 2, 3, 4, etc.

Secondly, to produce the negative ORP shift concurrent with polymerization, the DC component introduced into the AC time varying electromagnetic ionic wave current in pulsating manner plays the role of producing the negative ORP shift. The rate of the negative ORP shift is proportionate to the increase of the DC component current, but the DC current does not surpass the AC current. No membrane or barrier between the emitter pairs is required in the invention, thus balanced amount of H⁺ and OH⁻ ions are introduced into water via the DC component current. Therefore, there is no significant change in pH of the beverage yet more H⁺ and OH⁻ ions can be introduced into the water. As a result, the pH of the beverage substantially remains constant when the negative ORP shift takes place in the beverage.

Normally, 6.24×10¹⁸ electron charges would be produced if one ampere of the DC component current is flowing through the fluid per second. This increases the number of OH⁻ and H⁺ ions in the water accompanied by the rearrangement of water clusters. More electron donating water or alcohol clusters are formed and the ORP will progressively shift to a more negative reading, yet no significant change in pH of the beverage.

However, if more OH⁻ and H⁺ are produced in balanced quantity but without the effect of the capacitive field of the pulsating AC time varying electromagnetic wave nature to produce the electron donating clustering arrangement, there is no shift in ORP or pH. This is similar to the case of DC electrolysis without membrane giving no change in pH or ORP. According to the invention, the strength of capacitive field of the pulsating AC time varying electromagnetic wave nature is provided to be proportionate to the DC component so as to produce the desirable negative ORP antioxidizing property for the beverage. Therefore, one feature of the invention is that the antioxidizing ORP shift is produced only when the right combination of the DC biased time varying electromagnetic wave generator, the coupling of the generator with the capacitive emitters to generate the capacitive field across the emitters, and the DC bias. Another feature of the invention is that the negative ORP shift caused by the treatment of the invention takes place while the pH of the beverage to be treated does not substantially change. This is desirable because the change of pH often results in change of the chemical properties and tastes of the beverage which is found in the prior art chemical addition method.

Although adding alkaline chemicals such as NaOH can straightly increase the number of OH⁻ electron donating ions in the water to produce the electron donating antioxidizing effect, it also affects the pH and introduces undesirable effects into the beverage. This is against the objective of the invention requiring no intervention by any chemical addition.

More importantly, after being subject to the treatment of the invention, the beverage is energized to have increased internal energy, and the internal energy can be stored in the beverage for a period of time until it reaches the blood stream as demonstrated in the simulation test hereinafter.

### Reducing Burning Sensation of High Alcohol Content Distillates

The burning/sting sensation of the high alcohol content distillates is due to the Vanilloid Receptor VR-1 on the throat membrane picking up the alcohol single molecules and sending signal to the human brain as “HOT” signal. When the distillate is aged for a long time in barrel, the alcohol monomers polymerization with other alcohols, polyphenols, aldehyde, acetic acid into larger polymerized polymers of alcohol would occur. These large polymers do not react well with VR-1, hence less signal is picked by VR-1, with a result of reduced “HOT” sensation or feeling.

In the barrel, the chemical energy differences between alcohol molecules, water, aldehydes, acetic acids would drive these molecules to polymerize. But the chemical energy differences are very small, therefore the polymerization proceeds at a slow rate and takes years or decades to age and produce taste improvement and less burning smoothness effect. But once the distillate is taken out from cask and bottled, there is no new supply of chemical energy source from the cask materials or external source, hence no further change in beverage's quality and property is effected.

The treatment of the invention is an improvement in this regard and is further input of energy into the distillate even after it is bottled. The bottled distillate such as whisky when flowing through the treatment chamber of the system of the invention is subject to both the effects of the capacitive field generated by the AC component and the biased DC component, and therefore is energized to activate and vibrate the O—H, hydrogen, and polar bond energy of the distillate content molecules. This provides the free energy required by the alcohol polymerization and can be stored for a period of time, hence permitting continuous polymerization even when the distillate is bottled or is not in the ageing environment.

The DC component and the ionic current produced by the time varying electromagnetic field also produce the secondary effect on the emitter surface. This includes further oxidation of alcohol to aldehyde to acetic acid and even ester. These new compounds produced in the further oxidation process also provide the needed compounds for further polymerization. This makes possible the further aging effect of bottled whisky and other high alcohol content distillates.

### Reducing Astringency of Beverage

Astringency is another issue of the beverages including wine, tea, coffee, or other herbal plant beverages. Reduction of the astringency for the beverages through the prior art technologies normally takes an extended period of time, for example, reduction of astringency for wine may be achieved by years or decades of aging in oak cask to allow the tannins monomer, wine protein, alcohol and polyphenol molecules to undergo complex polymerization.

Similar to the reduction of throat burning sensation, the beverages receiving the treatment of the invention allows for the instant polymerization processes, especially tannins are polymerized with the protein in grapes or other organic compounds. These polymerization processes, especially the water clusters rearrangement, produce more electron donating clustering polymers, hence the astringency feel of the beverages is reduced in a very short time without the need of adding suppressing agent. For example, less sugar is used for ice lemon tea yet the same sweetness of tea is maintained. The astringency of wine especially young and bottled wine, after being treated by the invention, is significantly reduced within minutes.

### Increasing Body Fat Burning Metabolism

To produce the fat burning metabolism effect, the liquid beverage must have elevated free energy level stored in order for the body to crank start the fat burning metabolism. Glucose glycolysis metabolism produces the energy which requires lower energy to crank start, and it is always taking place preferentially over fat or protein metabolism. Fat burn metabolism is a more complex process and requires the higher energy. It only takes place either when glucose is consumed or when the body free energy is elevated to a higher level to start the fat burning, such as after a long distance of slow walking or jogging. When the body is at rest, the fat burning metabolism generally does not take place, or if any, is minimum, which may cause fat accumulation.

In the prior art, time varying electromagnetic wave induced by external inductor coils without direct contact with the beverage cannot elevate the free energy level of the beverage, and also the rate of increasing the vibration of the beverage is too slow to produce the instantaneous result.

Unlike the prior art, the system of the invention does not employ any inductor coil, instead only very closely packed emitter pairs are immersed and in direct contact with the beverage. With the correct frequency range, duty cycle, balance of DC and AC components of the generated capacitive field, vibration of O—H bonds can take place almost immediately. More importantly, the generated energy can be stored in the liquid beverage for a period of time before it is completely dissipated to the surroundings, thereby achieving the fat burn metabolism result together with other treatment effects discussed herein.

The improvement in fat burning metabolism effect with the liquid beverage treated by the invention will be demonstrated in the examples hereinafter.

### Reducing Harmful Toxic Effect After Alcohol Consumption

When alcohol enters the blood stream through the intestine wall, it would reach the liver first. The liver recognizes alcohol as a toxin and breaks it down by the following two-step process.

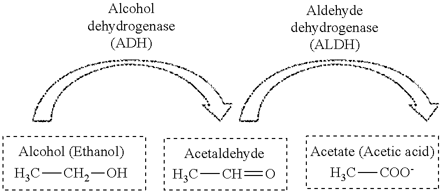

If the reaction is complete to reach acetic acid, it is harmless as acetic acid is non-toxic. However, the reaction may be incomplete if insufficient dehydrogenase enzyme is available, or inadequate free energy supply to the reaction to proceed fully to the acetic acid end product. Such incomplete conversion results in accumulation of acetaldehyde which is harmful to liver.

As described above, the treatment of the invention increases the free energy of the alcoholic beverage by elevating the vibration energy of polar bonds which can be stored for a period of time. It is found that the treated beverage is also capable of promoting the conversion of acetaldehyde to acetic acid, which is described in the simulation test hereinafter.

### Facilitating Ageing Effect of Alcoholic Beverage

Nowadays, alcoholic beverages cannot be aged once they are taken out from casks and bottled, because there is no new supply of chemical energy source from the cask materials or external source, hence no further change in beverage's quality and property takes place. Under the combined effect of the DC component and the ionic current in water produced by the pulsating capacitive field, the vibration and rotational internal energy of water clusters and dissolved ions are increased. The increased energy is stored in the beverage after the DC biased time-varying frequency pulsating electromagnetic wave treatment of the present invention. This excited energy has a long lasting residual effect, potentially up to a number of weeks, depending on the energy drain or dissipation in the system. This makes possible the further ageing effect of bottled whisky and other high alcohol content distillates, and therefore improves the quality and property of the alcoholic beverage after being taken out of the ageing storage environment.

### EXPERIMENTAL TESTS

### Example 1

### Simulation Test on Antioxidizing Effect of Treated Water

The amount of stomach acid HCl secreted every day is about 1.5 liters and the average pH of the stomach acid is about 2.5. The amount of water consumed by an adult is about 1.5 to 2 liters every day. The simulation test therefore uses 200 mL of pH 2.21 HCl to mix with 200 mL of water treated by the system of the invention and compared with the untreated water.

The acidic water after passing through the stomach is neutralized by NaHCO₃ secreted by the duodenum to approximately pH 8. This is simulated by adding 55 mL of NaHCO₃ to bring the pH back to 8.

The resulting ORP for the treated and untreated water are then measured by the ORP meter to confirm whether they are still antioxidizing before the water reaches the intestine for absorption into the blood stream.

Results are listed in Table 1.

TABLE 1

| | Untreated water | Treated water |
|---|---|---|
| Start (mouth) | pH = 7.31 | pH = 8.06 |
| | ORP = +282 mV | ORP = −254 mV |
| Stomach | pH = 2.65 | pH = 2.54 |
| | ORP = +345 mV | ORP = −72 mV |
| Duodenum | pH = 7.91 | pH = 8.01 |
| | ORP = +207 mV | ORP = −149 mV |

The test results reveal that the treated water maintains the antioxidizing state at the end of the test and the untreated water is oxidizing throughout the test.

### Example 2

### Fat Burning Metabolism Effect of Treated Beverage

To confirm whether the human body is undergoing carbohydrate metabolism or fat burn metabolism, an approach is to measure CO₂ content in the exhale breath. This can be carried out in the metabolic measurement chamber in hospital through CO₂ detection. The underlying principle is that more CO₂ is released per mole of oxygen in the inhale breath when burning carbohydrates/glucose, whereas less CO₂ is exhaled if the metabolism is through fat burn. The simplified glucose and fat oxidation is illustrated in the following.

When glucose is oxidized,
C₆H₁₂O₆+6 O₂→6 CO₂+6 H₂O+Energy
RQ (reactions quotient)=6 CO₂/6 O₂=1.0

When fat is oxidized,
C₁₆H₃₂O₂+23 O₂→16 CO₂+16 H₂O+Energy
RQ=16 CO₂/23 O₂=0.7

Thus the outcome is reduced CO₂ production for every oxygen molecule consumed.

Since the O₂ breathing in is approximately constant per breath, by measuring the CO₂ in the exhaling breath it is possible to know whether it is a glucose oxidation metabolism or a fat oxidation metabolism.

In this example, instead of using the hospital metabolic chamber, a portable Lumen® metabolic CO₂ sensor measurement meter is used for the test verification purpose. Measurement results provided by the Lumen metabolic CO₂ meter is scaled in 1 to 5. Reading shift towards 1 indicates more fat burn metabolism, while the shift towards 5 represents more carbohydrate burn metabolism. Detailed description of the Lumen sensor can be found in the website: https://www.lumen.me/how-it-works.

Test samples were collected from multiple subjects before and after consuming the designated beverages as shown in Table 2 below.

The typical sample tests results are shown in Table 2.

TABLE 2
Promotion in fat burning metabolism

| | No. of | | | | |
|---|---|---|---|---|---|
| | hours | Body | CO₂ level | CO₂ level | |
| Sample | after | temperature | before | after | Beverage |
| No. | meal | (C) | drinking | drinking | consumed |
| 1. | 1.5 | 36.3 | 5 | 3 | Treated |
| | | | | | coffee |
| 2. | 1.5 | 36.3 | 5 | 5 | Untreated |
| | | | | | coffee |
| 3. | 1.5 | 36.2 | 5 | 5 | Untreated |
| | | | | | Water 250 mL |
| 4. | 1.5 | 36.4 | 5 | 4 | Treated water |
| | | | | | 250 mL |
| 5. | 1.5 | 36.4 | 5 | 4 | Treated water |
| | | | | | 500 mL |
| 6. | 1.5 | 36.2 | 5 | 4 | Treated water |
| | | | | | 500 mL |
| 7. | 1.5 | 36.4 | 5 | 3 | Treated water |
| | | | | | 500 mL |
| 8. | 0.5 | 36.3 | 5 | 3 | Treated water |
| | | | | | 500 mL |

Before consuming the water and coffee treated by the system of the invention, the body is at sedentary condition and the Lumen CO₂ sensor reads 5 indicating carbohydrate metabolism. After consuming the treated water or coffee, the Lumen CO₂ sensor reads 4 or 3 indicating more fat burning metabolism is taking place.

After consumption of the untreated water and coffee, the Lumen CO₂ reading remains at 5, indicating no change in metabolism and the body is still undergoing carbohydrate metabolism.

As can be seen, all test samples show the same trend results, which proves the fat burn metabolism effect obtained by the treatment of the invention.

### Example 3

### Simulation Test for Reducing Toxic Acetaldehyde in Liver

To simulate the afore-mentioned conversion from alcohol to acetaldehyde and then to acetic acid, dehydrogenase taken from the mother of vinegar was added into the untreated whisky and the whisky treated by the system of the invention. The simulation test is focusing on determining the reaction conversion trend. The amount of enzyme used in this simulation test from the mother of vinegar is at a low level to simulate the condition of inadequate dehydrogenase.

Whisky is a common high alcohol content. A Johnny Walker Red Label whisky is used as the sample to better reflect the actual drinking situation.

The test results are listed in Table 3 below.

TABLE 3

| | | Untreated | Treated |
|---|---|---|---|
| | Whisky without | Whisky with | Whisky with |
| | Dehydrogenase | Dehydrogenase | Dehydrogenase |
| | addition | addition | addition |
| pH | 3.9 | 3.4 | 3.4 |
| Conductivity | 35.4 | 188.3 | 199.3 |
| (μS/cm) | | | |
| Acetaldehyde (ppm) | 174 | 185 | 150 |
| Acetic Acid (ppm) | 74.4 | 2,028 | 2,090 |

The test results show the following:
a) Acetaldehyde level (ppm) increased from 174 ppm for whisky without addition of dehydrogenase to 185 ppm for the same whisky after dehydrogenase addition simulating the situation in liver when the untreated whisky is drunk. This increase in acetaldehyde shows that the toxic acetaldehyde is accumulating in a condition that dehydrogenase is inadequate when whisky is not treated.
b) When the treated whisky is added with the same amount of dehydrogenase, the toxic acetaldehyde reduced from 174 ppm to 150 ppm. This result shows that the toxic acetaldehyde in the treated whisky is reduced in the liver.
c) The difference in acetaldehyde between the untreated and treat whisky is 35 ppm (185-150 ppm). This 35 ppm reduction in acetaldehyde content is a strong evidence of the treatment effect imposed by the invention to contribute the higher free energy to accelerate the liver alcohol to acetic acid conversion reaction with the result of reduced negative toxic health impact of alcoholic beverages.

The invention thus provides a system and a method for treating non-alcoholic and alcoholic beverages using an electromagnetic field comprising AC and DC components, which is very simple, relatively inexpensive and more environmentally sound than prior art technologies; and which is effective in providing various beneficial treatment effects in one go. The treatment effects cover the following.
1) Converting oxidizing alcoholic beverages into antioxidizing as a healthier alcoholic beverage is unique especially the conversion of high alcoholic content distillates to antioxidizing is new to the industry.
2) The ability to maintain the antioxidizing property of the beverage until it reaches the blood stream after consumption.
3) The capability of promoting fat burn metabolism without medicines or chemical addition is not known an any prior art.
4) The capability of reducing the harmful accumulation of acetaldehyde in liver by accelerating the complete conversion of alcohol to acetic acid instead of stopping at the incomplete intermediate alcohol to acetaldehyde reaction.
5) To simultaneously upgrade the beverage to possess various beneficial treatment effects in one go treatment, which has never been achieved before.
6) The ability to further reduce the burning sensation and improve the beverage quality of bottled high alcohol content distillates. This disrupts the industrial norm.
7) The instant treatment time in a few minutes to produce all the desirable effects is unique. The usual treatment time especially for alcoholic beverages take years to happen. This instant treatment makes possible to treat at home, commercial F&B outlets, and industrial users' instant treatment time requirement.
8) The same and single one system of the invention can be configured to treat a great variety of beverages from low electrical conductivity vodka, whisky to water/tea/coffee to high conductivity wine, juices, and coconut water etc. The variation in conductivity is from 10 μS/cm to 6,000 μS/cm.
9) Antioxidizing ORP of the treated alcoholic and non-alcoholic beverages can be preserved for long lasting shelf life, and reduced astringency and burning sensation are permanent.

Preferences and options for a given aspect, feature or parameter of the invention should, unless the context indicates otherwise, be regarded as having been disclosed in combination with any and all preferences and options of all other aspects, features and parameters of the invention.

While the embodiments described herein are intended as an exemplary system and method, it will be appreciated by those skilled in the art that the present invention is not limited to the embodiments illustrated. Those skilled in the art will envision many other possible variations and modifications by means of the skilled person's common knowledge without departing from the scope of the invention, however, such variations and modifications should fall into the scope of this invention.

1. A system for treating a liquid beverage using an electromagnetic field comprising alternating current (AC) and direct current (DC) components, comprising:
a device for generating a DC biased time-varying frequency pulsating electromagnetic wave comprising a biased DC component,
a power supply for supplying power to the device,
a treatment chamber containing a treatment liquid beverage in either static or flowing state,
one or more capacitive emitter pairs arranged in a predetermined pattern and placed to be in direct contact with the treatment liquid beverage, wherein each two adjacent capacitive emitters are spaced at a predefined distance and respectively electrically coupled with first and second output terminals of the device,
wherein the device is configured to apply the DC biased time-varying frequency pulsating electromagnetic wave to the treatment liquid beverage to generate a pulsating capacitive field having a field strength between the capacitive emitters, and
wherein a time-varying frequency and pulsating AC wave current comprising the biased DC component is provided in the treatment liquid beverage to produce a negative oxidation reduction potential (ORP) shift in the treatment liquid beverage whose pH substantially remains constant under a combined effect of the field strength of the pulsating capacitive field and the biased DC component.

2. The system of claim 1, wherein the time-varying frequency and pulsating AC wave current and the DC component of the generated field are provided to energize the treatment beverage such that one or more treatment effects are induced in the treatment liquid beverage.

3. The system of claim 2, wherein the treatment effects comprise one or more of the following:
1) reducing throat burning sensation of the treatment liquid beverage,
2) reducing astringency of the treatment liquid beverage,
3) increasing body fat/lipid burn metabolism through consumption of the treatment liquid beverage;
4) reducing harmful toxin effect of the treatment liquid beverage if alcoholic component is present, and
5) facilitating ageing effect of the treatment liquid beverage if alcoholic component is present.

4. The system of claim 1, wherein a plurality of emitter pairs are included and placed in the treatment chamber in a predetermined pattern and come in direct contact with the treatment liquid beverage, or the plurality of emitter pairs are mounted inline at a position where the emitters are in direct contact with the treatment liquid beverage.

5. The system of claim 1, wherein the emitters are provided in the form of plates or rods arranged in parallel coupling electrically with the first and second output terminals of the device generator, respectively, and wherein the emitters substantially extend over an entire length of the treatment chamber.

6. The system of claim 1, wherein the plurality of emitter pairs are provided as intermediate discharge emitters which are arranged in rows in parallel to each other, and wherein each of the emitters has a part of emitter surface covered by a conductive coating, and the coated emitter surfaces being oriented in one direction and arranged between non-coated emitter surfaces.

7. The system of claim 1, wherein the two adjacent capacitive emitters are spaced at a distance of less than 10 mm, preferably less than 6 mm.

8. The system of claim 1, wherein the device comprises an AC wave generator for generating an AC electromagnetic wave having a time-varying frequency at a desired sweeping time, and a DC biasing unit electrically coupled in series with the AC wave generator, wherein the DC biasing unit is configured to produce a DC output transmitted to the AC wave generator for generating the DC biased time-varying frequency pulsating electromagnetic wave comprising a biased DC component.

9. The system of claim 1, wherein the device comprises an AC wave generator for generating an AC electromagnetic wave having a time-varying frequency at a desired sweeping time, the AC wave generator being programmed to create imbalance time varying frequency wave form to generate the DC biased time-varying frequency pulsating electromagnetic wave comprising a net DC component.

10. The system of claim 1, further comprising an ORP meter for real-time monitoring and measuring the ORP of the treatment liquid beverage.

11. The system of claim 1, further comprising a FTIR spectrometer for scanning multiple consecutive beverage samples taken from the treatment liquid beverage to obtain a plurality of FTIR spectra which are mapped and analyzed to acquire a maximum vertical variation of peak height of a same absorbance peaks so as to determine a degree of energization, preferably a degree of non-thermal energization of the treatment liquid beverage.

12. The system of claim 1, further comprising a cooler used to cool down the treatment liquid beverage and/or a stirrer for homogenization of the treatment liquid beverage.

13. The system of claim 1, wherein the device is configured to generate the DC biased time-varying frequency pulsating electromagnetic wave comprising a biased DC component, having one or more of:
a wave form selected from square, sine or triangular wave,
a frequency between about 100 Hz and 1,000,000 Hz preferably between 500 to 10,000 Hz,
a sweeping frequency between 1 Hz and 1000 Hz, preferably 10 Hz and 100 Hz, and
a maximum peak voltage of the biased DC component has less than half of max AC peak voltage.

14. A method for treating a liquid beverage using an electromagnetic field comprising alternating current (AC) and direct current (DC) components, comprising the steps of:
generating a DC biased time-varying frequency pulsating electromagnetic wave comprising a biased DC component, and
applying the DC biased time-varying frequency pulsating electromagnetic wave to a treatment liquid beverage in either static or flowing state to provide a time-varying frequency and pulsating AC wave current comprising the biased DC component,
wherein the time-varying frequency and pulsating AC wave current comprising the biased DC component is transmitted to one or more capacitive emitter pairs arranged in a predetermined pattern and placed to be in direct contact with the liquid beverage so as to generate a pulsating capacitive field having a field strength between the capacitive emitters, thereby to produce a negative oxidation reduction potential (ORP) shift in the treatment liquid beverage whose pH substantially remain constant under a combined effect of the field strength of the pulsating capacitive field and the biased DC component.

15. The method of claim 14, comprising the step of measuring a conductivity of the liquid beverage for selecting the emitters adapted for the treatment of the liquid beverage.

16. The method of claim 14, further comprising the step of determining a degree of energization, preferably a degree of non-thermal energization, of the treatment liquid beverage, as a feedback control for the beverage treatment.

17. The method of claim 16, wherein the determination step comprises:
taking a plurality of consecutive samples from the treatment liquid beverage,
obtaining a plurality of FTIR spectra which represent respectively the plurality of consecutive samples,
mapping and analysis of the plurality of FTIR spectra for a same absorbance peak to collect information on peak height of the same absorbance peak, and
determining a maximum vertical variation of the peak height based on the lowest and highest peak heights of the same absorbance peak,
wherein the maximum vertical variation of the peak height reflects the degree of energization of the treatment liquid beverage.

18. The method of claim 17, wherein OH bond absorbance peak is mapped and analyzed.

19. The method of claim 14, wherein the time-varying frequency and pulsating AC wave current and the DC component of the generated field are provided to energize the treatment beverage such that one or more treatment effects are induced in the treatment liquid beverage.

20. The method of claim 19, wherein the treatment effects comprise one or more of the following:
1) reducing throat burning sensation of the treatment liquid beverage,
2) reducing astringency of the treatment liquid beverage,
3) increasing body fat/lipid burn metabolism through consumption of the treatment liquid beverage;
4) reducing harmful toxin effect of the treatment liquid beverage if alcoholic component is present, and
5) facilitating ageing effect of the treatment liquid beverage if alcoholic component is present.

21. The method of claim 14, further comprising the step of real-time monitoring and measuring the ORP of the treatment liquid beverage.

22. The method of claim 14, wherein each two adjacent capacitive emitters are spaced at a distance of less than 10 mm, preferably less than 6 mm.
